(19) United States
(12) Patent Application Publication
(10) Pub. No.: US 2006/0155168 A1
(43) Pub. Date: Jul. 13, 2006

(54) Optical snake

(76) Inventor: Alfred A. Pease, Ann Arbor, MI (US)

Correspondence Address: BUTZEL LONG, 350 SOUTH MAIN STREET, SUITE 300, ANN ARBOR, MI, 48104, US

(21) Appl. No.: 11/032,275

(22) Filed: Jan. 10, 2005

(51) Int. Cl. A61B 1/00; A61B 1/04

(52) U.S. Cl. 600/131; 600/109; 600/104

(57) ABSTRACT

An integrated solid state device for viewing and manipulating objects located in remote sites and locations which includes a flexible cable having a camera on one end and a viewer on an opposite end. The camera captures images near the end of the flexible cable and converts the images into a video signal that is transferred through wires that extend through the length of the flexible cable. The video signals are received by the viewer and converted into a displayed image. Objects are manipulated by a gripper that is provided at the end of the flexible cable that includes the camera. Operation of the gripper is controlled at the opposite end of the flexible cable

### TECHNICAL FIELD

The present invention relates to an optical device for observing remote sites and locations that have limited access. More specifically, the present invention is directed to an optical device that can be used for observing and manipulating objects in remote sites and locations which optical device can include different integral components and configurations.

### BACKGROUND ART

When inspecting the interior of engine cylinders at automotive equipping plants, repair shops, etc., a borescope is commonly inserted through a spark plug hole and used to inspect the interior of a cylinder without going through all the trouble of removing the engine's cylinder head(s). Borescopes are also used in the aviation industry, in marinas and shipyards, and in many other facilities in which various mechanical machines and devices are fabricated, assembled and/or repaired. Similar optical scopes are used to inspect the interior of scuba and gas storage tanks. Other fields in which borescopes or similar optical scopes are used include plumbing, mining, rescue, monitoring, and police investigations, to name only a few.

Borescopes and similar optical scopes commonly use cables that are formed from coherent bundles of optical fibers. Coherent bundles mean that the spatial relationship of individual each fiber to all the other fibers has to be maintained at each end of the cable. Since each fiber is essentially a “pixel,” it is necessary to maintain the spatial relationship of the fibers. This requirement contributes to the high cost of borescopes and similar optical scopes that use optical fiber cables.

In borescopes and similar optical scopes a lens is provided at the front end of the fiber bundles which focuses an image of what is being observed onto the polished ends of the fibers in the fiber bundle. A view lens is provided at the opposite end of the fiber bundle which transfers the relatively course image produced at the front end of the device to an observer using the device.

When using borescopes and similar optical scopes objects in dark areas illumination is commonly provided by means of a strong and relatively expensive halogen light source projected down the cable from the observation end. The light source used for illumination has to be strong due to the fact the light has to be transmitted from one end of the cable to the other and onto an object to be imaged and then the image has to be transmitted back through the cable to be observed.

The present invention provides devices for observing and manipulating objects in remote sites and locations which optical devices include a variety of integral components and configurations.

### DISCLOSURE OF THE INVENTION

According to various features, characteristics and embodiments of the present invention which will become apparent as the description thereof proceeds, the present invention provides an optical device for observing a remote situs which includes:

a length of flexible cable having a distal end and a proximal end;

an imager coupled to the distal end of the flexible cable for obtaining an image beyond the distal end of the flexible cable and converting the image into an electrical video signal;

a handheld image viewer coupled to the proximal end of the flexible cable for receiving the electrical video signal and converting the electrical video signal into a viewable image; and

electrical leads that extend between the distal and proximal ends of the flexible cable to transfer the electrical video signal from the imager to the image viewer.

The present invention further provides an optical device for observing and manipulating objects at a remote situs that includes:

a length of flexible cable having a distal end and a proximal end;

an imager coupled to the distal end of the flexible cable for obtaining an image beyond the distal end of the flexible cable and converting the image into an electrical video signal;

a device for manipulating objects located near the distal end of the flexible cable;

a handheld image viewer coupled to the proximal end of the flexible cable for receiving the electrical video signal and converting the electrical video signal into a viewable image, the handheld image viewer including a housing that houses a mechanism for activating the device that manipulates objects; and

electrical leads that extend between the distal and proximal ends of the flexible cable to transfer the electrical video signal from the imager to the image viewer.

### BRIEF DESCRIPTION OF DRAWINGS

The present invention will be described with reference to the attached drawings which are given as non-limiting examples only, in which:

FIG. 1 is a perspective view of an optical device according to one embodiment of the present invention.

FIG. 2A is a cross-sectional view of the handheld viewer of FIG. 1.

FIG. 2B is a cross-sectional view of an alternative handheld viewer similar to that of FIG. 1.

FIG. 3 is a perspective cross-sectional view of the flexible cable for an optical device according to one embodiment of the present invention.

FIG. 4 is a perspective view of an alternative viewing device for the proximal end of an optical device according to one embodiment of the present invention.

FIG. 5 is a perspective view of the distal end of an optical device according to one embodiment of the present invention that includes a gripper mechanism.

FIG. 6A is a perspective view of the gripper mechanism according to FIG. 5 in an open position.

FIG. 6B is a perspective view of the gripper mechanism according to FIG. 5 in a closed position.

### BEST MODE FOR CARRYING OUT THE INVENTION

The present invention is directed to a solid state optical snake device for observing and/or manipulating objects in remote sites or locations which can integrally incorporate a number of different components. The reference to “solid state” as used herein is intended to distinguish the present optical device from known optical devices that include optical fiber bundles as discussed above. Rather than use an optical fiber bundle, the optical device of the present invention uses a flexible cable that provides support for a number of integral attachments on the distal end thereof. In addition, the flexible cable provides a conduit for, or otherwise contains, a plurality of electrical leads that are used to electrically couple various devices at either end of the cable. In addition, to the electrical leads, optional fluid delivery tubes, pneumatic and/or hydraulic lines and/or wires or cables can extend through the flexible cable to affect the activities at the distal end of the optical device as discussed in more detail below. The distal end of the cable is configured to be integrally connected to an imager and a number of different components or devices. In addition to CMOS and CCD imagers or other types of cameras that are monochrome, color or infrared sensitive may be used. Additional exemplary components or devices include grippers or other article capturing devices, including hooks, baskets, electromagnets, etc., cutters, boring devices and other tools, thermocouples and other temperature sensing devices, chemical sensors, electromagnetic radiation detectors, and other devices. Further, radio transmitters that send out a locating signal may be used at the distal end of the device.

The proximal end of the flexible cable is provided with a display by which the user can observe the view at the distal end of the flexible cable and any structures or objects therein. In addition, the proximal end of the flexible cable can optionally be provided with a video connection such as a video output jack into which auxiliary display/viewing devices, image recorders, etc. can be connected. The proximal end of the flexible cable can further be provided with any suitable data connector port by which various devices which process electrical signals, including data recording devices can be coupled to the optical device. When sensors, monitors and/or detectors are used at the distal end of the flexible cable, signal processors together with displays and/or data recording devices, including computers can be coupled to the data connector port to receive and analyze data.

When grippers or other article capturing devices, cutters, boring devices and other tools are used at the distal end of the flexible cable various manipulators, trigger mechanisms, control devices, etc. provided at the proximal end if the flexible cable can be used to operate the grippers or other article capturing devices, cutters, boring devices and other tools. For example hand-held controllers that generate and forward electrical control signals through the electrical leads in the flexible cable can be used. Alternatively, devices that generate pneumatic or hydraulic pressure through fluid lines in the flexible cable can be provided at the proximal end of the flexible cable to operate grippers or other article capturing devices, cutters, boring devices and other tools. In a further alternative devices that generate mechanical forces through wires or solid cables that extend through the flexible cable can be coupled to the proximal end of the cable and used to operate the grippers or other article capturing devices, cutters, boring devices and other tools are used at the distal end of the flexible cable.

According to one embodiment of the present invention, an articulated gripper is designed and configured to be integrally coupled to a distal end of the flexible cable together with a CMOS or other camera so that the operation of the gripper can be observed.

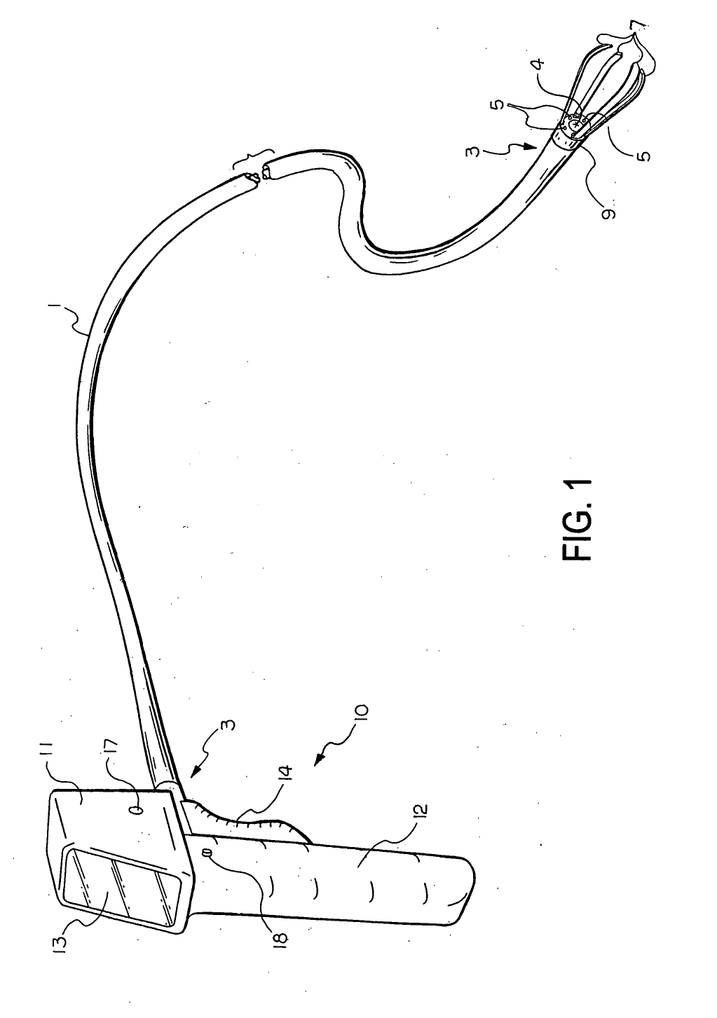

FIG. 1 is a perspective view of an optical device according to one embodiment of the present invention. The optical device of the present invention includes a flexible cable 1 having a distal end 2 and a proximal end 3. The flexible cable 1 is sufficiently rigid to allow it to be pushed though confined areas such as piping, conduits, walls, etc. The flexible cable 1 can be made from a plastic material and can either be solid (with channels for electrical wires, device actuation wires or cables, hydraulic and/or pneumatic lines as discussed below) or hollow with sufficiently thick walls to provide a desired degree of flexibility/rigidity. For a flexible cable 1 made of plastic, the flexible cable 1 can include any manner of embedded wires or a wound spring element that provides a desired degree of flexibility/rigidity. It is also within the scope of the present invention to make the flexible cable 1 from a hollow wound spring element with or without an outer coating layer. The flexible cable 1 can be provided with an outer coating of a suitable plastic material that will reduce friction in order to allow the flexible cable to slide easily in a confined area, and/or a plastic material which will prevent the flexible cable 1 from scratching or marring adjacent surfaces that are contacted during use.

The distal end 2 of the flexible cable 1 depicted in FIG. 1 is provided with housing 9 that contains and supports a lens 4, one or more LEDs 5 and gripper mechanism 6 that includes a set of gripper arms 7 that extend out from the housing 9. Inside the housing 9 is an imager 8 upon which light reflected off objects within the field of view of the lens 4 is focused. The imager 8 converts the received image into electrical data (video signal) that is transmitted though electrical wires that extend throughout the length of the flexible cable 1 to a display or image viewer located at the proximal end 3 of the flexible cable 1. The LED(s) is/are also coupled to electrical wire(s) that extend through the length of the flexible cable 1 and are coupled to a suitable battery or batteries and activation switch located at the proximal end 3 of the flexible cable 1. The power supply wires also supply electrical power from the battery/batteries to the imager 8 at the distal end 2 of the flexible cable 1.

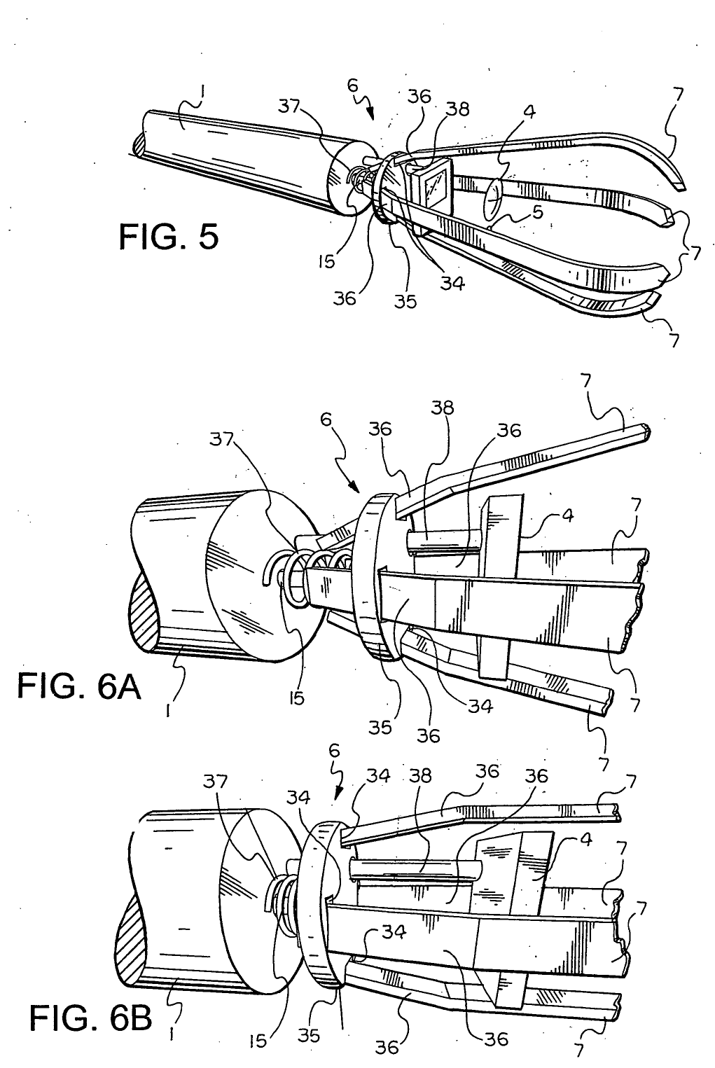

The housing 9 further houses a mechanism that operates the gripper arms 7 which are discussed in more detail in reference to FIGS. 5, 6A and 6B, below.

The length of the flexible cable 1 can vary according to the manner and environment in which the optical device is to be used. For most general household uses the flexible cable 1 can be from about 4 to 5 feet in length or longer. For commercial use the flexible cable 1 can be much longer than 5 feet, with lengths of from about 10 to 25 feet and more being within the scope of the present invention. The diameter of the flexible cable 1 can be from about 0.3 to 0.5 inches. Larger diameters can be used for some applications if desired and suitable.

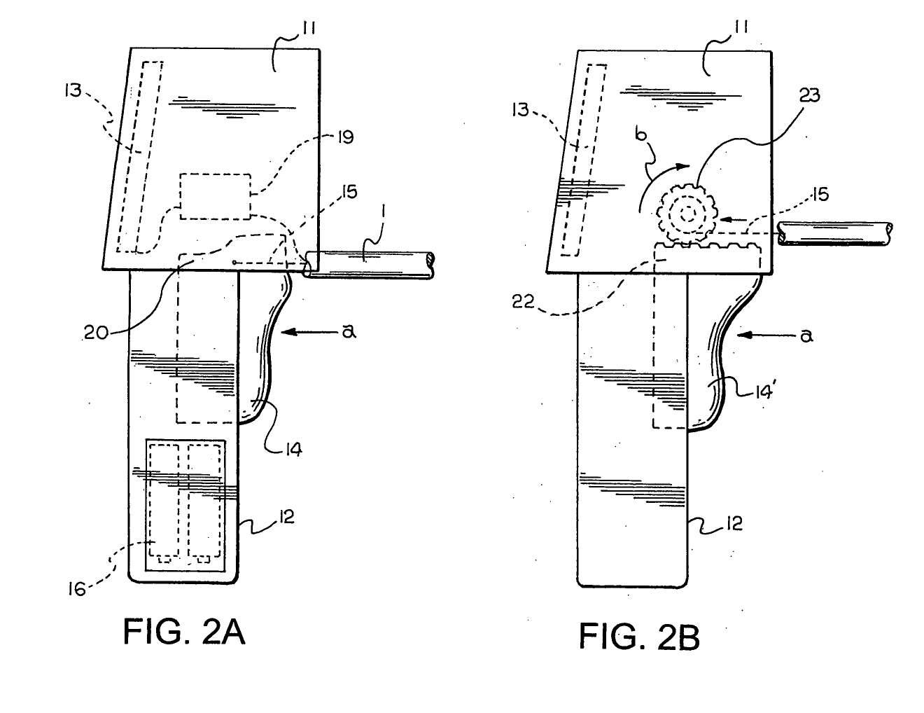

The proximal end 3 of the flexible cable 1 is coupled to an image viewer 10. In the embodiment of the invention shown in FIG. 1 the proximal end 3 of the flexible cable 1 is coupled to a handheld viewer 10 that includes a housing 11 that includes a hand grip 12 that is configured to be gripped and held by the user's hand. The housing 11 further supports a display screen 13 upon which the image generated by the imager at the distal end 2 of the flexible cable 1 is displayed. The display screen 13 can be a LCD or a CRT or other type of display with a LCD being particularly suitable for battery operation. The housing 11 depicted in FIG. 1 also includes a trigger 14 which can be operated by a user's finger. The trigger 14 is coupled within the housing 11 to an actuation wire or cable 15 which operates the gripper mechanism 6 at the distal end 2 of the flexible cable 1. The housing 11 further contains one or more replaceable or rechargeable batteries 16 (See FIG. 2A) for illuminating the LED(s) at the distal end 1 of the flexible cable 1 and powering the imager 8 and display screen 13. An auxiliary video output jack 17 can be provided on the housing 11 and used to patch the video signal to another display and/or recording device. Also shown in FIG. 1 is a lock button 18 which can be slid or depressed to lock the trigger 14 in a depressed position.

FIG. 2A is a cross-sectional view of the handheld viewer of FIG. 1. As shown, the trigger 14 is spring biased and configured to move linearly within handle or hand grip 12 when depressed in the direction of arrow “a.” The movement of the trigger 14 can be guided by cooperating structural elements between the trigger 14 and housing 11 such as block 20 on the top of trigger 14 which moves in a slot formed in the sides of housing 11. The proximal end 3 of actuation wire or cable 15 which operates the gripper mechanism 6 at the distal end 2 of the flexible cable 1 is coupled to block 20 of trigger 14 so that as trigger 14 is depressed in the direction of arrow “a” the proximal end 21 of actuation wire or cable 15 is pulled in the direction of arrow “b.” In FIG. 2A the display driver support and associated circuitry is indicated by reference numeral 19.

The trigger mechanism shown in FIG. 2A is merely one example of a trigger mechanism that can be used according to the present invention. In another embodiment depicted in FIG. 2B the trigger mechanism can use a linearly movable trigger 14′ that includes a toothed rack 22 that cooperates with a toothed pinion or toothed cam or toothed wheel 23 which rotates and pulls the actuation wire or cable 15. As depicted in FIG. 2B when trigger 14′ is depressed, toothed rack formed on the top of the trigger 14′ moves in the direction of arrow “a” which causes toothed wheel 23 to rotate in the direction of arrow “b.” As a result, the actuation wire or cable 15 which is coupled to toothed wheel 23 moves in the direction of arrow c.

In another embodiment a scissor-type trigger mechanism can be used in place of a linear movable trigger. Such a scissor-type trigger mechanism is that disclosed in U.S. Pat. No. 5,752,973. A pivotal trigger could also be used as an alternative to a linearly movable trigger. In an alternative to pulling the actuation wire or cable 15 the trigger mechanism could be configured to push the actuation wire or cable 15 to activate the gripper mechanism 6.

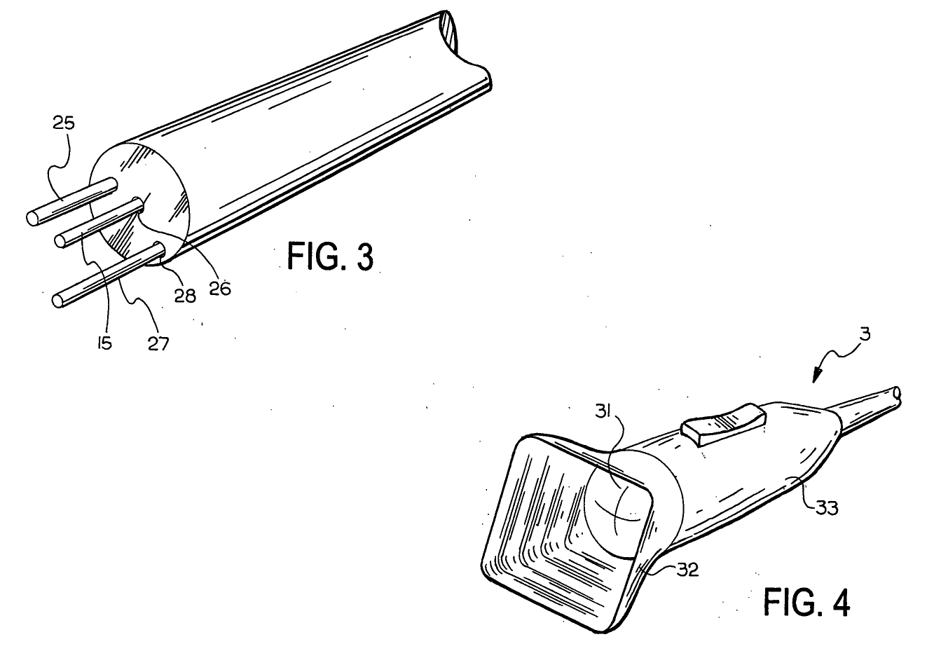

FIG. 3 is a perspective cross-sectional view of the flexible cable for an optical device according to one embodiment of the present invention. The flexible cable 1 depicted in FIG. 3 is a solid plastic cable that includes an embedded wire bundle 25 having individual wires for electrical power for illuminating one of more LED's 5 provided at the distal end 2 of the flexible cable 1, wires for supplying power to the imager 8, and wires for transferring the image data or video signals from the imager 8 at the distal end 3 of the flexible cable 1.

FIG. 3 also depicts a channel 26 through which an actuation wire or cable 15 can be passed and used to operate the gripper mechanism 6 as discussed herein. In addition, FIG. 3 depicts a wire or cable 27 that passes through channel 28 that can be used to articulate the distal end 2 of flexible cable 1. The wire or cable 27 extends through the length of flexible cable 1 in channel 28 and has a distal end that is attached in a fixed manner to or near the distal end 2 of the flexible cable 1 at or near one side thereof. When the proximal end of wire or cable 27 is pulled, the distal end 2 of the flexible cable 1 will bend or articulate to accommodate the effective shortening of wire or cable 27. This ability to bend or articulate will allow user to change the field of view of the lens 4 at the distal end 2 of the flexible cable 1. By rotating the flexible cable 1 about its axis (by manipulation of the proximal end 3) and articulating the distal end 2 a user can obtain a complete view of the area surrounding the distal end 2 of the device and any object therein. It is noted that the proximal end of the wire or cable 27 can be pulled using a mechanism similar to the trigger mechanisms discussed above. It is also possible to provide a mechanism that pushes the proximal end of the wire or cable 27 to articulate the distal end 2 of the flexible cable 1.

It is also possible to provide the flexible cable 1 shown in FIG. 3 with hydraulic and/or pneumatic lines that can be used to operate gripper mechanisms and other devices provided at the distal end of the device. In one embodiment a fluid passageway is provided so that a liquid or gaseous fluid can be dispensed from an outlet or nozzle provided at the distal end 2 of the flexible cable 1. For example, a sealing or insulating material could be dispensed from the distal end 2 of the flexible cable 1 by squeezing or applying pressure to a reservoir of fluid at or coupled to the proximal end 3 of the flexible cable 1.

FIG. 4 is a perspective view of an alternative viewing device for the proximal end of an optical device according to one embodiment of the present invention. FIG. 4 depicts a viewing device 30 that includes a lens 31 that is within a flexible eyepiece 32 that a user can hold against one eye to observe a display such as a LCD or a micro display that is secured within housing 33. Housing 33 also contains one or more replaceable or rechargeable batteries for illuminating one or more LED's provided at the distal end 2 of the flexible cable 1 and for supplying power to the imager 8 at the distal end 2 of the flexible cable 1 and to the display within housing 33. The viewer depicted in FIG. 4 can be used in an integral optical device according to the present invention that does not include a gripper mechanism. Alternatively, the viewer depicted in FIG. 4 can be used in an integral optical device that includes a gripper mechanism 6 in which case the housing 33 could be provided with squeezable trigger having a straight or curved tooth rack that rotates a toothed pinion or toothed cam or wheel which rotates and pulls the actuation wire or cable 15 as discussed above. Alternatively, a separate trigger mechanism could be separately coupled to the flexible cable 1 in front of the viewer housing 33 depicted in FIG. 4, as could a fluid dispenser.

FIG. 5 is a perspective view of the distal end of an optical device according to one embodiment of the present invention that includes a gripper mechanism. The gripper mechanism 6 includes a plurality of gripper arms 7 which extend through slots 34 provided in a gripper actuation cam 35. The gripper arms 7 are configured e.g. curved or bent at their proximal ends 36 and secured longitudinally so that when the gripper actuator cam 35 is moved along the axial direction of the flexible cable 1 it slides along the gripper arms 7 causing them to open and close.

FIG. 6A is a perspective view of the gripper mechanism according to FIG. 5 in an open position. In FIG. 6A the gripper actuator cam 35 has been moved in a forward position and the gripper arms 7 have been moved to their open position (as shown in FIG. 5) due to the configuration of the proximal ends 36 of the gripper arms 7.

FIG. 6B is a perspective view of the gripper mechanism according to FIG. 5 in a closed position. In FIG. 6B the gripper actuator cam 35 has been moved in a rearward position and the gripper arms 7 have been moved to their closed position (for gripping an object) due to the configuration of the proximal ends 36 of the gripper arms 7.

The gripper actuator cam 35 is biased by spring element 37 to normally (i.e. absent any other force) maintain the gripper arms 7 in their open position as shown in FIGS. 5 and 6A. The force of spring element 37 can also bias or assist in biasing the position of the trigger at the proximal end 3 of the flexible cable 1. Actuation wire or cable 15 is connected to the gripper actuator cam 35 so that pulling on the actuation wire or cable 15 as discussed above causes the gripper actuator cam 35 to move rearward against the force of the spring element 37 and move the gripper arms 7 to their closed position as shown in FIG. 6B. FIG. 5 depicts the relative position of an imager 8, LED 5 and lens 4 which are all supported in housing 9 as discussed above. FIGS. 6A and 6B depict the imager 8 and electrical wires 38 which transmit the image data or video signals to the proximal end 3 of the flexible cable 1. The imager 8 can be a CMOS or a CCD imager other type of imager device and may be monochrome, color or infrared sensitive.

It is to be understood that for purposes of the present invention the gripper mechanism 6 is not limit to that shown in FIGS. 5, 6A and 6B. Other gripper mechanisms 6 can be used, including pivoting scissor-type mechanism in which one or both arms are pulled, other cam mechanism similar to the gripper of U.S. Pat. No. 5,752,973 to Kieturakis.

It is also to be understood that, in addition to grippers, other article capturing devices, including hooks, baskets, electromagnets, etc. could be coupled to the distal end of the flexible cable and operated from the proximal end of the device, or even static hoods or baskets could be used. In addition, cutters, rams, rotors and other devices could be coupled to the distal end of the flexible cable and operated from the proximal end of the device using actuation wires or cables or hydraulic or pneumatic pressure or electrical power. Moreover, thermocouples and other temperature sensing devices, chemical sensors, electromagnetic radiation detectors, and other devices could be coupled to the distal end of the device and used according to the present invention.

Although the present invention has been described with reference to particular means, materials and embodiments, from the foregoing description, one skilled in the art can easily ascertain the essential characteristics of the present invention and various changes and modifications can be made to adapt the various uses and characteristics without departing from the spirit and scope of the present invention as described above and as set forth in the attached claims.

What is claimed is:

1. An optical device for observing a remote situs which comprises
a length of flexible cable having a distal end and a proximal end;
an imager coupled to the distal end of the flexible cable for obtaining an image beyond the distal end of the flexible cable and converting the image into an electrical video signal;
a handheld image viewer coupled to the proximal end of the flexible cable for receiving the electrical video signal and converting the electrical video signal into a viewable image; and
electrical leads that extend between the distal and proximal ends of the flexible cable to transfer the electrical video signal from the imager to the image viewer.

2. An optical device for observing a remote situs according to claim 1, wherein the imager comprises a CMOS or a CCD.

3. An optical device for observing a remote situs according to claim 1, wherein the image viewer includes a housing having a first portion that supports a display screen and a hand grip that extends from the first portion.

4. An optical device for observing a remote situs according to claim 3, wherein the display screen comprises a LCD or CRT.

5. An optical device for observing a remote situs according to claim 1, wherein the image viewer includes a video output jack.

6. An optical device for observing a remote situs according to claim 1, wherein the image viewer includes a battery compartment for holding at least one battery.

7. An optical device for observing a remote situs according to claim 1, wherein the image viewer includes a lens and an eyepiece.

8. An optical device for observing a remote situs according to claim 1, wherein the distal end of the flexible cable is articulated.

9. An optical device for observing a remote situs according to claim 1, further comprising a mechanism for manipulating objects located near the distal end of the flexible cable.

10. An optical device for observing a remote situs according to claim 9, wherein the mechanism is a gripper that is coupled to a housing located at the distal end of the flexible cable which housing houses the imager.

11. An optical device for observing a remote situs according to claim 10, further comprising a housing at the proximal end of the flexible cable that houses the image viewer and a manipulator for actuating the gripper.

12. An optical device for observing a remote situs according to claim 11, wherein the manipulator comprises a trigger which is coupled to a wire or cable that extends through the flexible cable and is coupled to the gripper.

13. An optical device for observing a remote situs according to claim 12, wherein the trigger is coupled to the wire or cable by a cam element.

14. An optical device for observing a remote situs according to claim 12, wherein the trigger moves linearly with respect to the housing.

15. An optical device for observing a remote situs according to claim 12, wherein the trigger moves pivotally with respect to the housing.

16. An optical device for observing a remote situs according to claim 11, wherein the image viewer comprises a LCD or CRT.

17. An optical device for observing a remote situs according to claim 11, wherein the housing of the image viewer includes a video output jack.

18. An optical device for observing a remote situs according to claim 1, further comprising a fluid conduit that extends through the flexible conduit.

19. An optical device for observing and manipulating objects at a remote situs that comprises:
a length of flexible cable having a distal end and a proximal end;
an imager coupled to the distal end of the flexible cable for obtaining an image beyond the distal end of the flexible cable and converting the image into an electrical video signal;
a device for manipulating objects located near the distal end of the flexible cable;
a handheld image viewer coupled to the proximal end of the flexible cable for receiving the electrical video signal and converting the electrical video signal into a viewable image, the handheld image viewer including a housing that houses a mechanism for activating the device that manipulates objects; and
electrical leads that extend between the distal and proximal ends of the flexible cable to transfer the electrical video signal from the imager to the image viewer.

20. An optical device for observing and manipulating objects at a remote situs according to claim 19, wherein the device for manipulating objects located near the distal end of the flexible cable comprises a gripper.

21. An optical device for observing and manipulating objects at a remote situs according to claim 20, wherein the gripper is coupled to the activating mechanism by a wire or cable that extends through the flexible cable.

22. An optical device for observing and manipulating objects at a remote situs according to claim 19, wherein the distal end of the flexible cable is articulated.

23. An optical device for observing and manipulating objects at a remote situs according to claim 19, wherein the handheld image viewer comprises a LCD or a CRT and the imager comprises a CMOS or a CCD.
